(19) United States
(12) Patent Application Publication
(10) Pub. No.: US 2020/0316157 A1
(43) Pub. Date: Oct. 8, 2020

(54) METHOD OF PROCESSING PLANT MATERIAL

(71) Applicant: Robert Ogilvie Watkins, Brisbane (AU)

(72) Inventor: Robert Ogilvie Watkins, Brisbane (AU)

(21) Appl. No.: 16/305,341

(22) PCT Filed: Mar. 21, 2017
(86) PCT No.: PCT/AU2017/050256

§ 371 Date: Nov. 28, 2018

(30) Foreign Application Priority Data
May 31, 2016 (AU) 2016902079

(51) Int. Cl. A61K 36/88; A61K 47/10; A23L 5/30; A23B 7/02; A23L 19/00; A23P 10/40

(52) U.S. Cl. CPC A61K 36/88; A61K 47/10; A23L 5/30; A23B 7/0215; A23V 2002/00; A23P 10/40; A61K 2236/13; A61K 2236/15; A61K 2236/17; A23L 19/01

(57) ABSTRACT

The present invention relates to a method of processing plant material to provide products such as food products, dietary supplements, pharmaceuticals, cosmetics and fertilizers. In particular, the invention relates to the processing of fruit and vegetable material to provide a product that can be used as a food source, dietary supplement or a pharmaceutical. Even more particularly, the invention relates to processing of green bananas such that the flesh and skin can be processed through separate streams.

### TECHNICAL FIELD

The present invention relates to a method of processing plant material to provide products such as food products, dietary supplements, pharmaceuticals, cosmetics and fertilizers. In particular, the invention relates to the processing of fruit and vegetable material to provide a product that can be used as a food source, dietary supplement or a pharmaceutical. Even more particularly, the invention relates to processing of green bananas such that the flesh and skin can be processed through separate streams.

### BACKGROUND ART

It is widely accepted that the prevalence of obesity in industrialised countries has been increasing. There are many factors leading to the increase in obesity, however, a significant factor is lack of energy balance, specifically, where energy intake (food and drinks) is greater than energy used (breathing, digesting, being physically active). Put simply, a combination of an abundance of high-calorie food and increasingly sedentary lifestyles, is contributing to what is being called an obesity epidemic.

In an effort to reverse the trend, government health departments have been undertaking public awareness campaigns encouraging people to look at their diets and also increase their levels of physical activity. This should have a flow-on effect of reducing the level of chronic disease and therefore reducing reliance on health services, with a concomitant reduction in economic burden.

One outcome of the public awareness campaigns is that people are taking more interest in their diet and are looking for better food choices, in terms of nutrient content. There has also been an increased interest in reducing intake of processed foods. In addition to high levels of fat and salt that can be found in some processed foods, the nutrient content of food can be reduced as a result of heat utilised in processing the food. For example, vitamin C is known to be destroyed at temperatures above 70° C.

A specific example of a food that can have reduced nutrient content as a result of heat treatment during processing is banana flesh, such as flesh from green bananas or from plantains. For example, WO 2004/069143 discloses a composition comprising fibre derived from plantains or bananas for use in the treatment of inflammatory bowel disease (IBD). One of the steps in the extraction of the soluble fibre is to boil the fruit in water for a defined period of time. Such a step would likely destroy heat-sensitive nutrients in the fruit. Whilst this might not be problem for the extraction of soluble fibre to treat IBD, it would certainly reduce the nutrient content, if a food product, medicinal product or dietary supplement was the desired outcome.

AU 2006233185 discloses a method of processing bananas to provide dietary fibre. The method includes subjecting diced banana flesh to a chemical wash at 70° C., and can also include a subsequent step of an acid or alkali treatment at elevated temperature (90° C.).

Recently, adherence to a ‘raw food diet’ has gained popularity. A raw food diet is a diet that consists predominantly of uncooked foods, and is considered by some to be the most natural and healthy diet. Generally to be considered ‘raw’, foods should not be subjected to a temperature higher than about 48° C. (about 118° F.). Followers of a raw food diet believe that subjecting foods to temperatures higher than about 48° C. (about 118° F.) results in loss of nutritional value.

It would therefore be advantageous to have a method of processing plant material to produce a food product, medicinal product or dietary supplement that retains the nutrient content of the plant material. In particular, it would be advantageous to have a method of processing green bananas or plantains that provided for separate processing streams for the skin and flesh. It would also be advantageous to have a method of processing whereby the nutrient content of the flesh was retained and/or the bioactive components of the skin were not destroyed.

It would furthermore be advantageous to produce a food product or dietary supplement that in addition to being nutritious, was also gluten-free. Such a food product or dietary supplement would cater for the increasing number of people with sensitivities to preservatives and/or gluten. There is also an increasing trend for people to want to consume products that have been minimally processed and are therefore considered more ‘natural’. It would therefore be advantageous to have a method of processing plant material to produce a gluten-free, nutritious, natural food product or dietary supplement.

It will be clearly understood that, if a prior art publication is referred to herein, this reference does not constitute an admission that the publication forms part of the common general knowledge in the art in Australia or in any other country.

### SUMMARY OF INVENTION

The present invention is directed to a method of processing green banana or plantain flesh, which may at least partially overcome at least one of the abovementioned disadvantages or provide the consumer with a useful or commercial choice.

With the foregoing in view, in a first aspect, the invention provides a method of processing green banana flesh, the method comprising the following steps:

(i) separating flesh and peel of a green banana; and

(ii) reducing the separated flesh to small particles,

wherein steps (i) and (ii) are undertaken at a temperature that would enable the flesh to retain raw food status.

In a second aspect, the invention provides a method of processing green banana flesh, the method comprising the following steps:

(i) separating flesh and peel of a green banana;

(ii) reducing the separated flesh to small particles; and

(iii) drying the small particles of banana flesh to produce a banana powder,

wherein steps (i) and (ii) are undertaken at a temperature that would enable the flesh to retain raw food status.

Bananas are traditionally classified into seven levels of ripeness, based on skin colour (Von Loesecke, H. W., 1950, Bananas, 2^{nd} Ed., Interscience, New York). Specifically, the classification is a colour scale with seven stages: 1, green; 2, light green; 3, half yellow-half green; 4, three-quarters yellow with green; 5, yellow with green tips; 6, full yellow; and 7, yellow with brown spots. The colour changes from stage 1 (green) through to stage 7 (yellow with brown spots) are a direct result of the reduction in chlorophylls a and b in the peel as the fruit ripens. Thus the colour changes are a visual indicator of the conversion of starch in the flesh of the banana to sugars.

In the context of the present invention, reference to a ‘green’ banana, is a reference to a banana that is at stage 1 or stage 2 as defined for the Von Loesecke colour scale. Once a banana ripens to stage 3 as defined in the scale, the starch content of the flesh is reduced, with concomitant increase in sugar content, to the extent that any resultant product (such as banana flour) would not have the desired nutritional or medicinal properties.

Preferably, the flesh and peel are separated using a device as described in Australian provisional patent application no. 2013900558. As described therein, the device comprises a pair of conveyor systems each including an elongate belt, the elongate belts of the pair of conveyor systems being aligned to form an upper conveyor system and a lower conveyor system, the respective elongate belts counter-rotating at different relative speeds, and spaced from one another by a separation distance, the separation distance between the elongate belts tapering to a pinch point to allow a banana to enter the pinch point and be at least partially crushed when passing the pinch point.

The device enables banana flesh to be separated from the banana skin efficiently, rapidly and safely, without requiring any heat treatment of the bananas. Furthermore, the device enables both the banana flesh and banana skin to be collected and processed through different processing streams.

Specifically, following passage of a banana through the pinch point, the flesh and peel are partially separated. Complete separation of flesh and peel is achieved by agitation of the flesh and peel in a vessel having an inlet, an outlet, and agitation paddles. The action of the agitation paddles on the skins dislodges residual pulp from the skins. The action of the agitation paddles on the pulp reduces the size of the pieces of pulp. Smaller pieces of pulp can reduce downstream processing time. The agitation means can additionally comprise vibration means which prevent the pulp and skins from sticking to the inside walls of the vessel. The vibration means also assist in directing the pulp and skins onto the agitation paddles. Following a period of time sufficient for dislodging residual pulp from the banana skins, the pulp and skins exit the vessel via the vessel outlet. Upon exiting the vessel, the skin and pulp can be further processed through a separation device.

A separation device is a further optional processing step for separating the skins and pulp into different processing streams. Specifically, the skins and pulp can be processed through a separation device, such as a sorting tunnel, which allows the pulp to proceed through processing into food products, medicinal products, dietary supplements or cosmetics, and the skins to pass into a different processing stream. The complete separation of skins and pulp into different processing streams can be achieved using a sieving device, sized with one or more differently sized meshes such that the banana pulp passes through but not the banana skins.

A particularly preferred sieving device is a trommel or rotary screen which is essentially a perforated cylindrical drum. Typically, the pieces of banana pulp are smaller than the pieces of banana skin, so the pulp passes through the screen apertures, whilst the skin exits the drum at the opposite end to which the combined skin and pulp are fed into the trommel screen.

Alternatively, the separation of pulp and skin can be achieved on a conveyor. That is, the pulp and skin can be transferred from the peeling machine to a conveyor, where they are separated, and from there continue through the appropriate processing stream.

Optionally, the tip of the banana comprising the flower remnant can be removed prior to separating the peel and flesh. The flower remnant and the tip can contain toxins, such as mould, and bacteria and other pathogens, therefore removing the tip avoids contamination, thereby retaining value in the final dried banana powder as a food product, medicinal product and/or dietary supplement.

Optionally, the bananas can be washed prior to removal of the tip of the banana comprising the flower remnant. The washing can simply comprise passage of the bananas through a trough containing a fluid suitable for cleaning extraneous material, such as bugs and sap, from the exterior of the bananas. Alternatively, the washing can comprise use of a spray nozzle system to spray a suitable cleaning fluid onto the exterior of the bananas, to remove extraneous material, such as bugs and sap, from the exterior of the bananas. The fluid can be any suitable solution for washing the bananas and can simply be water (including hot water or steam). Alternatively, the fluid can be any suitable sanitation solution such as those known in the fruit and vegetable processing industry. For example, the sanitation solution can be hydrogenised (either acid or alkaline) water, a mild solution of chlorine, or a weak solution of hypochlorite. Preferably, the mild solution of chlorine is an aqueous solution comprising chlorine within the range 100-1500 ppm. The mild solution of chlorine can therefore comprise 100, 200, 300, 400, 500, 600, 700, 800, 900, 1000, 1100, 1200, 1300, 1400 or 1500 ppm chlorine. For embodiments where the sanitation solution is a weak solution of hypochlorite, preferably this is an aqueous solution comprising hypochlorite within the range 100-1500 ppm. The aqueous solution can therefore comprise 100, 200, 300, 400, 500, 600, 700, 800, 900, 1000, 1100, 1200, 1300, 1400 or 1500 ppm hypochlorite.

Where the fluid is other than water, the bananas can be subjected to a rinsing step after the washing step to remove traces of the washing fluid from the bananas. The rinsing step simply consists of rinsing the bananas with water. The rinsing can be achieved using any suitable means. Typically, the rinsing is achieved by spraying water on the bananas, or by passing the bananas through a rinsing trough containing water.

Following separation of the peel and flesh, the flesh is essentially in the form of a mix of unevenly sized particles of flesh. From such a mix, the separated flesh can be reduced to smaller, evenly sized particles, by any suitable means. For example, the unevenly sized particles of flesh can be homogenized to a pulp using an industrial blender or chopping machine. In a preferred embodiment, the unevenly sized particles of flesh are passed through two stages of micronizing. The first stage consists of a process that essentially dices the unevenly sized particles of flesh into evenly sized particles of about 0.2-1.0 cm³. The second stage of the micronizing process results in the formation of a slurry consisting of just flesh or flesh and water. Importantly for downstream processing of the slurry, the flesh particles in the slurry are similarly sized. In a particularly preferred embodiment, the second stage of the micronizing process results in particles of banana flesh that are sized between about 100 and about 800 microns.

Optionally, if the resultant slurry contains too much liquid, the excess liquid can be removed by filtering or straining the slurry, or by decanting the excess liquid. Other methods for removing excess liquid, and thereby obtaining a slurry with a desired moisture content are known in the art, and are encompassed. Such methods include centrifuging the slurry, using dry air, such as freeze-dried air which can have as low as 1% moisture, using mildly heated air, or gently heating the slurry.

The resultant particles of flesh can be utilized as a food product or food additive. Alternatively, the resultant particles of flesh can be dried prior to use as a food product, food additive or dietary supplement.

The resultant particles of flesh can be dried using any suitable means known in the art for drying food products. Preferred methods for drying include evaporation to dryness under ambient conditions or using heat to assist with the evaporation, a refractance window drying process (RWDP) which uses mild heating conditions, or drying on a drying bed under ambient or heated conditions. Reducing the particles to be sized within the range 100-800 microns enables the particles to be dried in a relatively short period of time. Advantageously, reducing the size of the particles to fall within the range 100-800 microns, allows the particles to be dried rapidly without excessive heating (and therefore without excessive energy consumption) and also without concomitant destruction of nutrients in the banana particles which would accompany excessive heating and slow drying.

Once the banana flesh has been dried, it can be used in many ways, for example, as a food product, a dietary supplement, a functional ingredient, in medicinal products or cosmetics, or as a source of dietary fibre. The dried banana flesh can be used as is, or alternatively, the dried banana flesh can be ground to a powder and the powder used as a food product, a dietary supplement, a functional ingredient, in medicinal products or cosmetics, or as a source of dietary fibre. In a particularly preferred embodiment, the dried banana flesh can be ground to a powder and used as a flour, including as an alternative to wheat flour. A particular use of the dried banana flesh is as a source of resistant starch, for example, in a dietary supplement. Alternatively, the dried banana flesh can be utilized in combination with a pharmaceutically acceptable carrier to provide a pharmaceutical composition. For example, the dried banana flesh can be added to an ointment base to provide a dried green banana flesh skin ointment.

Resistant starch is a type of fibre that is resistant to breakdown in the stomach and small intestine. It is therefore able to pass through to the large intestine where it provides a food source for gut flora. Resistant starch provides metabolic benefits, such as improved insulin sensitivity and lower blood sugar levels, but is only found in limited foods, such as green bananas and raw potatoes. Given that these are foods that don't feature heavily in diets, a dietary supplement is the most convenient form in which to increase intake of resistant starch.

It is particularly important that the steps of separating the flesh and peel of bananas, and reducing the flesh to small particles are undertaken at a temperature that would enable the flesh to retain raw food status. Typically, the flesh will qualify as raw food if it has not been heated to greater than about 48° C. (about 118° F.). Therefore, it is preferable that the steps of separating the flesh and peel of green bananas, and reducing the flesh to small particles are undertaken at temperatures within the range 15° C.-48° C. The steps of separating the flesh and peel of bananas, and reducing the flesh to small particles can therefore be independently undertaken at 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47 or 48° C.

Preferably the steps of separating the flesh and peel of green bananas, and reducing the flesh to small particles are independently undertaken at a temperature less than about 30° C. In particularly preferred embodiments of the invention, the steps of separating the flesh and peel of green bananas, and reducing the flesh to small particles are independently undertaken at a temperature selected from 21° C. or 22° C.

Following separation of the peel and flesh, the peel can be dried used any suitable drying apparatus known in the industry. Preferably, the peel is dried in a dehydrator for about 5-15 hours, preferably about 10-12 hours. In order to maintain the activity of components in the peel, the temperature at which drying is undertaken should not exceed about 48° C. In a particularly preferred embodiment, the peel is dried at about 45° C. The peel can be dried and be processed similarly to the green banana flesh by micronising followed by drying.

Following drying, the dehydrated peel can be ground to give a green banana skin powder. The green banana skin powder has a multitude of diverse uses due to its nutritional and mineral content, and its medicinal properties. For example, the green banana skin powder can be used in the preparation of a skin ointment, a nutritional supplement or a fertilizer.

Preferably, the processing time for the green banana peel is kept to a minimum as the fresh peel contains a sticky sap that can interfere with the processing steps. Advantageously, minimizing the processing time for the banana peel minimizes the risk of contamination of the resultant green banana skin powder.

Alternatively, following separation of the peel and flesh, the green banana peel can be diced and stored in a pharmaceutical grade liquid. The pharmaceutical grade liquid can be any suitable liquid, including 95% pharmaceutical grade ethanol, propanediol, or glycerine. The green banana peel can be stored in the pharmaceutical grade liquid for a suitable period of time to maximise extraction of components in the skin into the pharmaceutical grade liquid. Typically, the green banana peel is stored in the pharmaceutical grade liquid for about 2, 3, 4, 5, 6, 7, 8, 9, 10, 11 or 12 weeks. For example, in one embodiment, the green banana peel can be stored in 95% pharmaceutical grade ethanol, propanediol or glycerine for about 8-12 weeks.

To avoid degradation of the skin components, the extraction is preferably undertaken at ambient temperature. The term ‘ambient temperature’ is intended to mean room temperature and therefore refers to temperatures within the range 15° C.-30° C. The extraction in 95% pharmaceutical grade ethanol, propanediol or glycerine can therefore be undertaken at 15, 16, 17, 18, 19, 20, 21, 22, 23, 24, 25, 26, 27, 28, 29 or 30° C.

Following extraction, the peel can be removed from the pharmaceutical grade liquid by filtration to give a green banana peel extract.

The resultant green banana peel extract can be used in the preparation of a skin ointment. The green banana peel extract has anti-inflammatory, antioxidant, and anti-fungal properties and therefore can be used generally to promote skin healing. For example, due to the components in green banana peel, a skin ointment prepared using green banana peel extract and an ointment base can be used to heal burns, cuts, skin abrasions, skin rashes and/or irritations, nappy rash or cold sores.

The present invention thus provides a method of processing green bananas that allows the flesh to be separated from the banana skin efficiently, rapidly and safely. The present invention also enables both the banana flesh and banana skin to be collected and processed through different processing streams. In particular, the banana flesh is subjected to further processing to provide a product that can be used as a food product, a dietary supplement, in pharmaceutical products or on cosmetics, whilst the banana skin is subjected to a separate processing stream to provide a product that can be used in a skin ointment or other pharmaceutical products, cosmetics or a dietary supplement.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawing, in which:

FIG. 1 is a flow chart showing processing steps in one embodiment of the current invention.

### DESCRIPTION OF EMBODIMENTS

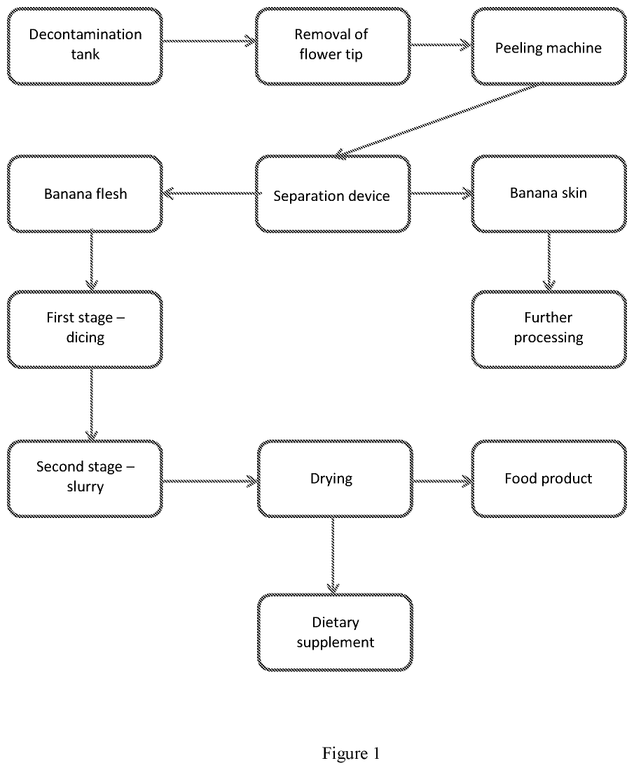

Referring to FIG. 1, which is a flow chart showing processing steps according to one embodiment of the current invention.

Step 1—Decontamination Tank

The first step comprises passage of bananas through a decontamination tank. The decontamination tank contains a fluid such as water or a mild sanitation solution, for cleaning extraneous material, such as bugs and sap, from the exterior of the bananas. Use of a mild sanitation solution in the decontamination tank also acts to disinfect and/or sterilize the bananas prior to subsequent processing.

Step 2—Removal of Flower Tip

The second step comprises removal of the tip of the banana comprising the flower remnant. The flower remnant and the tip can contain toxins, such as mould, and bacteria and other pathogens, Removing the tip avoids contamination of the final product, whether that be a food product, medicinal product, cosmetic or dietary supplement from the banana flesh, or a product derived from the banana skin. In addition, the flower tip can be very hard, and if not removed can create problems in subsequent processing of the banana flesh.

The tip can be removed by any suitable means, including manually, or automated. Where the tips are to be removed manually, the bananas can be placed on a conveyor belt for passage of the bananas past process workers. Alternatively, a conveyor belt can be used to transport the bananas through a cutting machine that utilizes cutting means to remove the tip.

Step 3—Peeling Machine

The third step comprises separating the peel and flesh using a device as described in Australian provisional patent application no. 2013900558. As described therein, the device comprises a pair of conveyor systems each including an elongate belt, the elongate belts of the pair of conveyor systems being aligned to form an upper conveyor system and a lower conveyor system, the respective elongate belts counter-rotating at different relative speeds, and spaced from one another by a separation distance, the separation distance between the elongate belts tapering to a pinch point to allow a banana to enter the pinch point and be at least partially crushed when passing the pinch point. Following passage of a banana through the pinch point, the flesh and peel are partially separated.

The device enables banana flesh to be separated from the banana skin efficiently, rapidly and safely, without requiring any heat treatment of the bananas. Furthermore, the device enables both the banana flesh and banana skin to be collected and processed through different processing streams.

For many downstream applications of the flesh or skin, it is particularly important that the separation of flesh and skin is undertaken without heat. It can be difficult to separate flesh from skin, especially if the bananas are green (under ripe). Prior art methods have overcome this difficulty by subjecting the bananas to hot water (usually within the range 50-90° C.). The hot water degrades the starch barrier between the skin and the flesh and therefore allows the skin to be more easily separated from the flesh. This is similar to the process that naturally occurs when a banana ripens. Specifically, as a banana ripens, the starch levels reduce so that a ripe banana is easier to peel than a green banana. The hot water also breaks down valuable components of the banana flesh, such as vitamins and nutrients. Furthermore the heat treated skins also contain less actives such as antioxidants, minerals and anti-inflammatory compounds than non-heat treated skins, and therefore also have reduced nutraceutical and pharmaceutical value. Preferably, the present method does not utilise heat to assist in peeling the bananas, and therefore retains the starch content of the flesh and actives content of the skin.

Alternatively, where the downstream applications for the flesh are not dependent on the vitamin and nutrient content, or for the skin are not dependent on the antioxidant, mineral and anti-inflammatory properties, the bananas can be subjected to hot water to assist with separation of the skin from the flesh.

Optional Step Between Step 3 and Step 4

An optional step, not shown in FIG. 1, which can take place between steps 3 and 4 is subjecting the combined skins and flesh to mechanical means to completely separate any flesh remaining on the skins. The mechanical means can operate by any suitable mechanism, including one or more of rotation, vibration and tumbling.

Step 4—Separation Device

The fourth step comprises separation of the skins and pulp into different processing streams. Specifically, the skins and pulp are processed through a separation device, such as a sorting tunnel, which allows the pulp to proceed through processing into food products, dietary supplements, medicinal products or cosmetics, and the skins to pass into a different processing stream. The complete separation of skins and pulp into different processing streams can be achieved using a sieving device, sized with one or more differently sized meshes such that the banana pulp passes through but not the banana skins.

A particularly preferred sieving device is a trommel or rotary screen which is essentially a perforated cylindrical drum. Typically, the pieces of banana pulp are smaller than the pieces of banana skin, so the pulp passes through the screen apertures, whilst the skin exits the drum at the opposite end to which the combined skin and pulp are fed into the trommel screen.

It is important that the separation device provides for both the flesh and peel to be separately collected, as the peel is also economically valuable. For example, banana peels are known to contain antioxidants, minerals and anti-inflammatory compounds.

Step 5—Dicing (Stage 1, Particle Size Reduction)

The fifth step consists of a process that essentially dices the unevenly sized particles of flesh into evenly sized particles of about 0.2-1.0 cm³. The process of dicing banana flesh can generate heat, which not only makes the banana flesh difficult to work with, but as mentioned previously, reduces the starch content of the flesh, and therefore the nutritional value of resultant food products and dietary supplements. It is particularly important that the dicing step (essentially the first stage of particle size reduction) is undertaken under conditions that don't generate heat. The dicing is preferably undertaken in a food-grade dicing machine through which the banana flesh moves by way of gravity feed. In a particularly preferred embodiment of the fifth step, the unevenly sized particles of banana flesh enter the dicing machine at an entry point which is higher than the exit point. The unevenly sized particles of banana flesh pass through the dicing machine under the effects of gravity, being diced into evenly sized particles of about 0.2-1.0 cm³ as they progress. The resultant evenly sized particles of banana flesh exit the dicing machine under the effects of gravity. The rate of passage of the flesh through the dicing machine is controlled so as not to generate heat. In addition, dicing banana flesh can create a sticky residue, which blocks the machine. Consequently, controlling the rate of passage of the flesh through the dicing machine is also necessary to reduce build up of sticky residue and therefore limit down time for cleaning the machine.

Step 6—Slurry (Stage 2, Particle Size Reduction)

The sixth step comprises a process of micronizing the evenly sized particles from the fifth step results in the formation of a slurry or a paste consisting of just flesh or flesh and water. Importantly for downstream processing of the slurry or paste, the flesh particles in the slurry are similarly sized. In a particularly preferred embodiment, the second stage of the micronizing process results in particles of banana flesh that are sized between about 100 and about 800 microns. This step can be considered to be a second stage of particle size reduction.

Banana pulp is a very difficult product to handle. Each variety has different characteristics. For example, the two most important varieties in Australia from a commercial perspective, Cavendish and Lady Finger, have pulp with vastly different characteristics. Pulp from Cavendish bananas is quite moist with a tendency to have a mucilaginous or ‘slimy’ texture. In contrast, pulp from Lady Finger bananas is drier and can be more starchy in nature, however, it does release moisture over time, if left to stand. It is therefore important that any methods for processing banana pulp can be readily adapted to suit the characteristics of the pulp, irrespective of the banana variety being processed. It is particularly important during the sixth step of the process of FIG. 1, where the evenly sized particles of flesh from the fifth step are micronized to form a slurry.

For example, if Lady Finger banana flesh, which has a drier or starchier texture is being processed, the sixth step can include the addition of water to the flesh during micronizing, to adjust the moisture content of the resultant slurry. However, as Lady Finger banana flesh releases moisture over time, the addition of water needs to be balanced with the length of time taken to micronize the flesh to a slurry. In addition, the passage of the flesh through the micronizing also needs to be monitored to ensure that it is not so rapid as to result in heating of the flesh.

In contrast, if Cavendish banana flesh is being processed, the flesh has a mucilaginous or ‘slimy’ texture due to its lower starch content than Lady Finger banana flesh. This results in a slurry which can be quite moist. The sixth step can therefore optionally include a process for adjusting the moisture content of the resultant slurry. For example, any food-grade product suitable for absorbing excess moisture can be added to the flesh during micronizing to adjust the moisture content of the resultant slurry. Alternatively, the moisture content of the slurry can be adjusted to a suitable level by filtering or straining the slurry, or by decanting the excess liquid. Other methods for removing excess liquid, and thereby obtaining a slurry with a desired moisture content include centrifuging the slurry, using dry air, such as freeze-dried air which can have as low as 1% moisture, using mildly heated air, or gently heating the slurry.

The passage of the flesh through the micronizing also needs to be monitored to ensure that it is not so rapid as to result in heating of the flesh. As for step 5, where dicing of the flesh proceeded through the dicing machine under the effects of gravity, the micronizing step preferably also proceeds under the effects of gravity to avoid heating the flesh, and also to control the rate of passage of the flesh through the micronizing step to reduce build up of sticky residue and therefore limit down time for cleaning the machine.

Step 7—Drying

In the seventh step of the process as set out in FIG. 1, the micronized particles of flesh are dried using any suitable means known in the art for drying food products. Such means can include using a tunnel dryer or a belt dryer. Preferred methods for drying include evaporation to dryness under ambient conditions, a refractance window drying process (RWDP) which uses mild heating conditions, or drying on a drying bed under ambient or heated conditions. The micronized particles of flesh can also be dried using freeze drying. In a particularly preferred embodiment, the micronized particles of flesh are dried using a high speed airflow fluid bed drying machine. Reducing the particles to be sized within the range 100-800 microns enables the particles to be dried in a relatively short period of time. Advantageously, reducing the size of the particles to fall within the range 100-800 microns, allows the particles to be dried rapidly without excessive heating and concomitant destruction of nutrients in the banana particles which would accompany excessive heating.

In certain applications of the invention, where the process is required to scaled up for high throughput, it becomes particularly important to ensure the micronized particles of flesh are dried as quickly as possible, without the use of excessive heat, so that the nutrient content is retained and not destroyed during the drying step. Methods for rapid, low temperature drying are known in the art and include the use of drum dryers, tunnel dryers and flaking drum dryers.

An alternative method for rapid, low temperature uses belt dryers which can incorporate microwaves, infrared technology, heated air, heated fluid bed, or simply a heated bed in order to dry the slurry. Where a belt dryer is utilized, it is particularly important that the drying be undertaken rapidly and without the use of excessive heat, so that the nutrient content of the slurry is retained. Application of a very thin (typically 1-10 mm) layer of slurry to the belt assists with ensuring the slurry can be dried quickly at relatively low temperatures, thereby retaining the nutrient content of the slurry in the resultant dried product. In the context of using a belt dryer, the phrase ‘relatively low temperatures’ is a reference to a temperature within the range 30° C.-80° C. Although the process has been broadly described above as involving seven steps, it will be appreciated that the process can be adapted or customized dependent on the desired product. For example, step 6 which relates to particle size reduction could be an optional step, such that following the particle size reduction of step 5, the diced banana flesh could immediately proceed to a drying step (step 7), resulting in a coarse product.

Alternatively, step 6 could be followed by an optional step 6A, whereby the particles produced during step 6 are subjected to an additional micronizing step to further reduce the particle size. Optional step 6A can be undertaken by any suitable device, such as an homogeniser, or a wet mill. Drying the particles of optional step 6A would result in a particularly fine powder.

In further alternatives, step 6 could be followed by an optional step 6B, or optional step 6A could be followed by optional step 6B. Optional step 6B reduces the moisture content of the slurry resulting from step 6 or optional step 6A, and could comprise filtering or straining the slurry in situations where due to the nature of the flesh being micronized, the resultant slurry contains too much liquid. Filtering or straining the slurry acts to remove excess liquid. Alternatively, optional step 6B could comprise decanting the excess liquid, centrifuging the slurry, using dry air, such as freeze-dried air which can have as low as 1% moisture, using mildly heated air, or gently heating the slurry.

Having broadly described the invention, non-limiting examples of products prepared using the method of the invention, will now be given.

Sample 1—Comparative Sample—Commercial Banana Flour from Sri Lanka

A commercially available banana flour, originating from Sri Lanka, was obtained for comparative testing with banana flour produced using the method of the present invention. The Sri Lankan banana flour was likely prepared using a traditional method involving the following steps: washing the green bananas in hot water, hand peeling, slicing the green banana flesh into slices or pieces and then air-drying the slices or pieces in the sun over a period of 12-24 hours.

Sample 2—Banana Flour

Green Cavendish bananas were passed through a tank containing a mild sanitation solution, to clean the exterior of the bananas. The flower tip was then removed from the green bananas, prior to passing the bananas through a peeling machine to separate the peel and flesh. The green banana flesh was then diced to give evenly sized particles of about 0.2-1.0 cm³. The evenly sized particles were then micronized to give a slurry of flesh, and the slurry was dried on a belt dryer, resulting in green Cavendish banana flour.

Sample 3—Green Banana Resistant Starch

Green Lady Finger bananas were passed through a tank containing a mild sanitation solution, to clean the exterior of the bananas. The flower tip was then removed from the green bananas, prior to passing the bananas through a peeling machine to separate the peel and flesh. The green banana flesh was then diced to give evenly sized particles of about 0.2-1.0 cm³. The evenly sized particles were then micronized to give a slurry consisting of flesh and water, and the slurry was dried on a belt dryer, resulting in green Lady Finger banana resistant starch.

Sample 4—Lady Finger Banana Skin Powder

Green Lady Finger peel from the process used to generate Sample 3 was dried in a dehydrator for 10-12 hours at about 45° C. The resultant dehydrated peel was then ground to give green Lady Finger banana skin powder.

Sample 5—Banana Skin Liquid Extract

Green Lady Finger peel from the process used to generate Sample 3 was diced and stored in 95% pharmaceutical grade ethanol for four weeks. After this time, the peel was removed by filtration to give a green Lady Finger banana skin extract.

Sample 6—Banana Ointment

Green Lady Finger banana skin extract from the process used to generate Sample 5 was mixed into an ointment base to produce green Lady Finger banana skin ointment.

Phytochemical Content

Each of the products detailed in Samples 1 to 6 were analysed by high performance liquid chromatography (HPLC) and liquid chromatography-mass spectrometry (LC-MS) for phytochemicals, such as chlorogenic acids, catechins, proanthocyanidins, biogenic amines (L-DOPA, dopamine, serotonin, serotonin derivatives, tryptophan), phytosterols and saponins. The results are set out in Table 1.

TABLE 1
phytochemical content of Examples

| Component | Sample 1 | Sample 2 | Sample 3 | Sample 4 | Sample 5 | Sample 6 |
|---|---|---|---|---|---|---|
| Chlorogenic acids | 1.9 | 6.1 | 4.0 | 1.8 | 129.3 | 0.8 |
| (mg/kg) | | | | | | |
| catechins | N/D | N/D | N/D | N/D | N/D | N/D |
| Flavonoids (as | 35.5 | 65.9 | 51.0 | 234.7 | 9.9 | N/D |
| rutin equivalents, | | | | | | |
| mg/kg) | | | | | | |
| Proanthocyanidins | 0.210 | 0.252 | 0.241 | 0.256 | 0.057 | 0.182 |
| (% w/w) | | | | | | |
| L-DOPA | N/D | N/D | N/D | N/D | N/D | N/D |
| Dopamine | N/D | N/D | N/D | N/D | N/D | N/D |
| Serotonin | N/D | N/D | N/D | N/D | N/D | N/D |
| Tryptophan | 9.7 | 43.2 | 30.2 | 30.7 | N/D | N/D |
| (mg/kg) | | | | | | |
| 5- | 2.6 | 53.2 | 132.1 | 30.0 | N/D | N/D |
| hydroxytryptophan | | | | | | |
| (mg/kg) | | | | | | |
| Total phytosterols | N/D | N/D | N/D | 0.2748 | 0.0299 | 0.0872 |
| (% w/w) | | | | | | |

N/D—not detected

From the above data, it can be seen that Samples 2 and 3 had a higher content of chlorogenic acid, flavonoids, tryptophan and 5-hydroxytryptophan than comparative Sample 1.

Sample 5 (liquid extract from banana skin) had the highest content of chlorogenic acid, whilst Sample 4 (lady finger banana skin powder) had the highest content of flavonoids and phytosterols.

Antimicrobial Activity

For each of the Samples 1 to 4 and 6, four extractions using different solvents were undertaken. Solvent (water, ethanol, acetone, hexane) (10 mL) was added to the sample (1 g), which was then vortexed, and sonicated (30 minutes). The resultant solution was centrifuged, decanted and evaporated to dryness. For Sample 5, a sample (10 g) was simply evaporated to dryness.

The resultant extracts were assessed for anti-microbial activity using a range of dilutions in a 96-well plate method. Specifically, the % inhibition of Escherichia coli (E. coli) and Staphylococcus aureus (S. aureus) were measured. The results are set out in Tables 2 and 3.

TABLE 2
% inhibition of E. coli

| | Product | | | | | | | | | | |
|---|---|---|---|---|---|---|---|---|---|---|---|
| | Sample 1 | | Sample 2 | | Sample 3 | | Sample 4 | | Sample 5 | Sample 6 | |
| Extract | % inhibition (extract concentration - g/mL) | | | | | | | | | | |
| Water | 33 | (0.17) | 0 | | 13 | (0.17) | 34 | (0.17) | | 22 | (0.17) |
| ethanol | 100 | (0.02) | 100 | (0.02) | 100 | (0.02) | 86 | (0.02) | | 100 | (0.02) |
| Acetone | 100 | (0.02) | 100 | (0.02) | 100 | (0.02) | 0 | | | 93 | (0.04) |
| Hexane dried | 100 | (0.02) | 100 | (0.04) | 100 | (0.02) | 26 | (0.17) | 0 | 100 | (0.02) |

TABLE 3
% inhibition of S. aureus

| | Product | | | | | | | | | | | |
|---|---|---|---|---|---|---|---|---|---|---|---|---|
| | Sample 1 | | Sample 2 | | Sample 3 | | Sample 4 | | Sample 5 | | Sample 6 | |
| Extract | % inhibition (extract concentration - g/mL) | | | | | | | | | | | |
| Water | 0 | | 0 | | 0 | | 0 | | | | 17 | (0.17) |
| Ethanol | 100 | (0.02) | 100 | (0.02) | 100 | (0.02) | 82 | (0.02) | | | 100 | (0.02) |
| Acetone | 100 | (0.02) | 100 | (0.02) | 100 | (0.02) | 0 | | | | 100 | (0.04) |
| Hexane | 100 | (0.02) | 34 | (0.04) | 100 | (0.02) | 90 | (0.17) | 100 | (0.83) | 100 | (0.02) |
| dried | | | | | | | | | | | | |

From the above data it can be seen that all of the Samples had some anti-microbial activity, with Samples 2, 3 and 6 having the greatest activity. Each of these Samples completely inhibited both E. coli and S. aureus at the lowest extract concentrations tested.

Hygiene

Samples 1 and 3 were subjected to procedures for determining the colony count of yeasts and moulds in accordance with Australian Standard AS 5013.29-2009 (publisher, Standards Australia). Additional analyses of samples 1 and 3 to determine aerobic plate count of bacteria in accordance with AOAC 990.12 and coliform and E. coli counts of bacteria from the coliform group, and separately count of E. coli in accordance with AOAC 991.14 (Association of Analytical Communities) were undertaken. The results are set out in Table 4.

TABLE 4
hygiene results

| | | | Standard | | |
|---|---|---|---|---|---|
| | Mould | Yeast | Plate Count | Coliforms | E. coli |
| | (CFU/g) | (CFU/g) | (CFU/g) | (CFU/g) | (CFU/g) |
| Sample 1 | 2,300 | 900 | ~600,000 | 900 | <10 |
| Sample 3 | <3 | <3 | <1,000 | <3 | <3 |

From the above data, it can be seen that Sample 3 clearly meets the strict standards required for food production. The method for production of banana flour of the present invention therefore results in a product that meets the hygiene standards required for food production with respect to levels of mould, yeast and bacteria.

The present invention thus provides a method for separating banana flesh from banana peel efficiently, rapidly and safely. The present invention also enables both the banana flesh and banana skin to be collected and processed through different processing streams. In particular, the invention relates to a method of processing banana flesh to provide a product that can be used as a food source, a dietary supplement, in pharmaceutical products or in cosmetics. Advantageously, the product is produced rapidly (from whole, green bananas to green banana powder in about 30 minutes) without the need for heat until the drying step (which would destroy nutrients in the flesh and actives in the skin), chemicals or other additives, such as preservatives. Importantly, the only time heat is used during the whole process is the use of heat during the drying step.

A further advantage of the present invention, is that the process is undertaken as a continuous in-line process, and is therefore a relatively rapid process. Thus, degradation of nutrients in the banana flesh is minimized, and difficulties associated with slow processing (banana flesh can become more difficult to work with over time) are also minimized.

Hygiene is a major issue with working with green bananas. Handling and environmental conditions can allow bacteria, moulds and other such pathogens to thrive and multiply extremely rapidly on banana flesh. A further advantage of the present process being a continuous in-line process and therefore relatively rapid, is that bacteria, moulds and other such pathogens do not have an opportunity to attack the banana flesh. Consequently, the process described herein results in a product that can be utilised raw in food products and dietary supplements, without the need for additives such as preservatives. The process avoids the hygiene risks associated with current methods for processing banana flesh and results in a natural, preservative- and gluten-free product that meets the strict guidelines for hygiene, food safety and production which exist around the world.

A further advantage of the present process being relatively rapid, is that oxidation of the banana flesh is minimized. If the period of time from peeling through to obtaining a dry product is too long, the banana flesh oxidizes resulting in a darkening in colour of the flesh and therefore the subsequent dried product. This is not only visually detracting, but can result in reduced nutrient content. Prior art methods use antioxidants such as citric acid and ascorbic acid to minimize the darkening of banana flesh with the aim of producing a visually appealing product. However, the process of the present invention does not require the use of such antioxidants, whilst resulting in a visually appealing product. Products prepared according to the method of the present invention therefore have retained value both in terms of nutrient content and avoidance of any additives such as antioxidants.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A method of processing green banana flesh, the method comprising the following steps:
(i) separating flesh and peel of a green banana; and
(ii) reducing the separated flesh to small particles,
wherein steps (i) and (ii) are undertaken at a temperature that would enable the flesh to retain raw food status.

2. The method of claim 1, wherein step (ii) comprises a first stage of particle size reduction resulting in particles of flesh of a first size between about 0.2 cm³ and 1.0 cm³, followed by a second stage of particle size reduction whereby the particles of flesh are further reduced to a second size of about 100 μm-800 μm.

3. The method of claim 1, wherein step (ii) is followed by step (iii) comprising drying the particles of flesh.

4. The method of claim 3, wherein each of steps (i), (ii) and (iii) is independently undertaken at a temperature within the range 15° C.-48° C.

5. (canceled)

6. (canceled)

7. The method of claim 4, wherein the resultant dried flesh is ground to a powder.

8. (canceled)

9. (canceled)

10. (canceled)

11. A method of processing green banana flesh, the method comprising the following steps:
(i) separating flesh and peel of a green banana;
(ii) reducing the separated flesh to small particles; and
(iii) drying the small particles of flesh to produce a green banana powder,
wherein steps (i) and (ii) are undertaken at a temperature that would enable the flesh to retain raw food status.

12. The method of claim 11, wherein step (ii) comprises a first stage of particle size reduction resulting in particles of flesh sized between about 0.2 cm³ and 1.0 cm³, followed by a second stage of particle size reduction whereby the particles of flesh are further reduced to a size of about 100 μm-800 μm.

13. The method of claim 11, wherein each of steps (i), (ii) and (iii) is independently undertaken at a temperature within the range 15° C.-48° C.

14. (canceled)

15. (canceled)

16. The method of claim 11, wherein the resultant dried flesh is ground to a powder.

17. (canceled)

18. (canceled)

19. (canceled)

20. A method of processing green banana peel, the method comprising the following steps:
(i) separating flesh and peel of a green banana; and
(ii) reducing the separated peel to small particles,
wherein steps (i) and (ii) are undertaken at a temperature that would enable the peel to retain raw food status.

21. The method of claim 20, wherein step (ii) comprises a first stage of particle size reduction resulting in particles of peel sized between about 0.2 cm³ and 1.0 cm³, followed by a second stage of particle size reduction whereby the particles of peel are further reduced to a size of about 100 μm-800 μm.

22. The method of claim 20, wherein step (ii) is followed by step (iii) comprising drying the particles of peel.

23. The method of claim 22, wherein each of steps (i), (ii) and (iii) is independently undertaken at a temperature within the range 15° C.-48° C.

24. (canceled)

25. (canceled)

26. The method of claim 20, wherein the resultant dried peel is ground to a powder.

27. (canceled)

28. (canceled)

29. (canceled)

30. The method of claim 20, wherein step (ii) is followed by step (iii) comprising adding the particles of peel to a pharmaceutical grade liquid to create a mixture of peel and pharmaceutical grade liquid.

31. The method of claim 30, wherein the pharmaceutical grade liquid is selected from the group consisting of 95% ethanol, propanediol and glycerine.

32. The method of claim 31, wherein the pharmaceutical grade liquid is 95% ethanol.

33. The method of claim 30, wherein the particles of peel are stored in the pharmaceutical grade liquid for a period of about 8 to about 12 weeks.

34. (canceled)

35. The method of claim 30, wherein the mixture of peel and pharmaceutical grade liquid is filtered to give a solution of green banana peel extract.

36.-56. (canceled)
